(19) United States
(12) Patent Application Publication
(10) Pub. No.: US 2009/0326224 A1
(43) Pub. Date: Dec. 31, 2009

(54) THIENO PYRIMIDINE COMPOUNDS

(75) Inventor: Aleem Gangjee, Allison Park, PA (US)

Correspondence Address: PEPPER HAMILTON LLP, ONE MELLON CENTER, 50TH FLOOR, 500 GRANT STREET, PITTSBURGH, PA, 15219, US

(73) Assignee: DUQUESNE UNIVERSITY OF THE HOLY SPIRIT, Pittsburgh, PA (US)

(21) Appl. No.: 12/438,764

(22) PCT Filed: Aug. 29, 2007
(86) PCT No.: PCT/US07/77082

§ 371 Date: Jun. 2, 2009

Related U.S. Application Data
(60) Provisional application No. 60/824,294, filed on Sep. 1, 2006.

(51) Int. Cl. C07D 495/04

(52) U.S. Cl. 544/278

(57) ABSTRACT

A compound for treating cancer tumors, particularly ovarian cancer tumors, is described, where a fused cyclic pyrimidine having a cancer treating ability is effective to allow selective delivery to a cancerous tumor.

This application claims priority to U.S. Provisional Application No. 60/824,924 filed Sep. 1, 2006 titled “Thieno Pyrimidine Compounds”, the contents of which are incorporated herein by reference.

Most cancer chemotherapy agents do not specifically selectively target cancer tumor cells; however, chemotherapy agents have targeted both normal and tumor cells. This lack of selectivity for tumor cells results in cytotoxicity to the normal cells and is also one of the major causes of chemotherapeutic failure in the treatment of cancer. Further, advanced stage and platinum resistant tumors may be difficult to treat with traditional chemotherapeutic agents such as, but not limited to, carboplatin or paclitaxel (docitaxel).

A type of folate receptor FR—FRα is overexpressed on a substantial amount of certain surfaces of a number of cancerous tumors including, but not limited to, ovarian, endometrial, kidney, lung, mesothelioma, breast, and brain tumors.

In most normal tissues, the FRα is not present. In most normal tissues, folic acid is not taken up by normal cells by way of a reduced folate carrier system (RFC). In light of the specificity of the folic acid, conjugates of folic acid have been used to selectively deliver toxins, liposomes, imaging and cytotoxic agents to FRα expressing tumors.

However, one of the major limitations of the foregoing, such as cytotoxic-folic acid conjugates, is that this requires cleavage from the folic acid moiety to release the cytotoxic drug. Even more importantly, premature release of the cytotoxic agent during the transport before reaching the tumor destroys selectivity and thereby leads to undesired toxicity in normal cells.

Further, since the folic acid moiety of the cytotoxic-folic acid conjugate is difficult to cleave, then the anti-tumor activity is hindered as a result of the inability or reduced ability to release the cytotoxic agent. Accordingly, treatment of the tumor cells with the cytotoxic agent is either hindered or rendered nil as a result of the difficulty in cleaving the cytotoxic agent moiety from the folic acid-based conjugate.

There remains a need for compositions that selectively target the FR of tumor cells.

### SUMMARY OF THE INVENTION

One embodiment of the present invention is a compound for selectively targeting FR, particularly FRα, of tumor cells. Such a compound may selectively target the GARFTase enzyme and the AICARFTase enzyme.

A further embodiment of the present invention provides a compound of the formula:

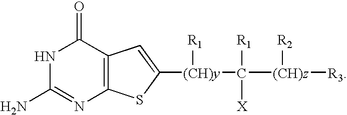

In another embodiment of the present invention, a compound of the formula:

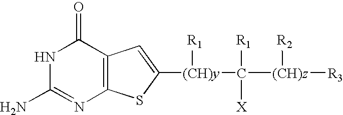

wherein

R1 is H, trifluoromethyl, trifluoromethyl ketone, formyl, methyl alcohol, methylamine or a bond;

X is an aroyl-L-glutamate group or H, wherein if X is H, R2 is an aroyl-L-glutamate group and if X is an aroyl-L-glutamate group, R2 is H or a bond;

R3 is H, trifluoromethyl, trifluoromethyl ketone, formyl, methyl alcohol or methylamine;

y is an integer between 0 and 6; and

z is an integer between 1 and 7, wherein the sum total of y and z is equal to or less than 7, and when the sum total of y and z is 1 or 2, X must be an aroyl-L-glutamate group is provided.

Another embodiment of the present invention provides a pharmaceutical composition comprising a compound of the formula:

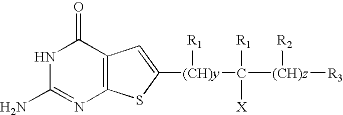

wherein

R1 is H, trifluoromethyl, trifluoromethyl ketone, formyl, methyl alcohol, methylamine or a bond;

X is an aroyl-L-glutamate group or H, wherein if X is H, R2 is an aroyl-L-glutamate group and if X is an aroyl-L-glutamate group, R2 is H or a bond;

R3 is H, trifluoromethyl, trifluoromethyl ketone, formyl, methyl alcohol or methylamine;

y is an integer between 0 and 6; and

z is an integer between 1 and 7, wherein the sum total of y and z is equal to or less than 7, and when the sum total of y and z is 1 or 2, X must be an aroyl-L-glutamate group is provided.

The pharmaceutical composition may comprise a pharmaceutically effective amount of the foregoing compound. The pharmaceutical composition may further comprise a pharmaceutically acceptable carrier and/or excipient.

A further embodiment of the present invention are methods of treating cancer and/or inhibiting tumor cell growth by administering a compound of the formula:

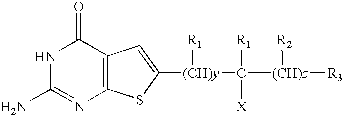

wherein

R1 is H, trifluoromethyl, trifluoromethyl ketone, formyl, methyl alcohol, methylamine or a bond;

X is an aroyl-L-glutamate group or H, wherein if X is H, R2 is an aroyl-L-glutamate group and if X is an aroyl-L-glutamate group, R2 is H or a bond;

R3 is H, trifluoromethyl, trifluoromethyl ketone, formyl, methyl alcohol or methylamine;

y is an integer between 0 and 6; and

z is an integer between 1 and 7, wherein the sum total of y and z is equal to or less than 7, and when the sum total of y and z is 1 or 2, X must be an aroyl-L-glutamate group is provided.

Another embodiment of the present invention is to provide effective delivery of a non-toxic FR targeting compound to the cancerous tumor for treating a patient.

A further embodiment of the present invention is to efficiently target a cancerous tumor by administering such compounds.

### BRIEF DESCRIPTION OF THE DRAWINGS

FIG. 1(a) shows one embodiment of a general chemical formula for a fused cyclic pyrimidine; and

FIG. 1(b) shows a another embodiment of the formula of FIG. 1(a), where n is the total number of CH₂ groups between the major cyclic/ring groups shown as I and II.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

Optical Isomers, Diastereomers, Geometric Isomers, Tautomers. Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

It must also be noted that as used herein and in the appended claims, the singular forms “a”, “an”, and “the” include plural reference unless the context clearly dictates otherwise. Thus, for example, reference to an “fibroblast” is a reference to one or more fibroblasts and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with elastin digest, can include, but is not limited to, providing an elastin digest into or onto the target tissue; providing an elastin digest systemically to a patient by, e.g., intravenous injection whereby the therapeutic reaches the target tissue; providing an elastin digest in the form of the encoding sequence thereof to the target tissue (e.g., by so-called gene-therapy techniques). “Administering” a composition may be accomplished by injection, topical administration, or by either method in combination with other known techniques. Such combination techniques include heating, radiation and ultrasound.

The term “animal” as used herein includes, but is not limited to, humans and non-human vertebrates such as wild, domestic and farm animals.

As used herein, the term “cancer” refers to any type of cancer, including, but not limited to, ovarian cancer, leukemia, lung cancer, colon cancer, CNS cancer, melanoma, renal cancer, prostate cancer, breast cancer, and the like.

The term “improves” is used to convey that the present invention changes either the appearance, form, characteristics and/or the physical attributes of the tissue to which it is being provided, applied or administered. The change in form may be demonstrated by any of the following alone or in combination: enhanced appearance of the skin; increased softness of the skin; increased turgor of the skin; increased texture of the skin; increased elasticity of the skin; decreased wrinkle formation and increased endogenous elastin production in the skin, increased firmness and resiliency of the skin.

The term “inhibiting” includes the administration of a compound of the present invention to prevent the onset of the symptoms, alleviating the symptoms or eliminating the disease, condition or disorder, for example, reducing growth/replication.

As used herein, the term “patient” refers to members of the animal kingdom including, but not limited to, human beings.

By “pharmaceutically acceptable”, it is meant the carrier, diluent or excipient must be compatible with the other ingredients of the formulation and not deleterious to the recipient thereof.

Unless otherwise indicated, the term “skin” means that outer integument or covering of the body, consisting of the dermis and the epidermis and resting upon subcutaneous tissue.

As used herein, the term “therapeutic” means an agent utilized to treat, combat, ameliorate, prevent or improve an unwanted condition or disease of a patient. In pant, embodiments of the present invention are directed to the treatment of cancer or the decrease in proliferation of cells.

A “therapeutically effective amount” or “effective amount” of a composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, “tumor” refers to an abnormal growth of cells or tissues of the malignant type, unless otherwise specifically indicated and does not include a benign type tissue. The tumor may comprise of at least one cell and/or tissue.

The present invention has filled the above described need and satisfied the above objects by providing a narrow range of compounds that selectively target the FR of tumor cells. Other folate receptors of the FR-beta type are overexpressed on surfaces of myeloid leukemia cancerous tumors. The term “FR” used herein includes receptors selected from the group consisting of FR-alpha, FR-beta and mixtures thereof. In a preferred embodiment, the compositions selectively target FR-alpha and beta of cancerous tumor cells.

Very significantly, the cancer-treating compound is not significantly taken up by a cell or tissue using the RFC system.

The cancer-treating agent is a fused cyclic pyrimidine and is used to selectively target FR of ovarian tumors, advanced stage cancerous tumors that express FR receptors and drug-resistant tumors such as, but not limited to, those resistant to carboplatin, paclitaxel, and/or docitaxel. The receptors are preferably FR-alpha and beta types,

In yet another related object, the compound will allow penetration into the cancerous cells expressing FR, that is FR-alpha and FR-beta, but not into a cell using the reduced folate carrier system (RFC).

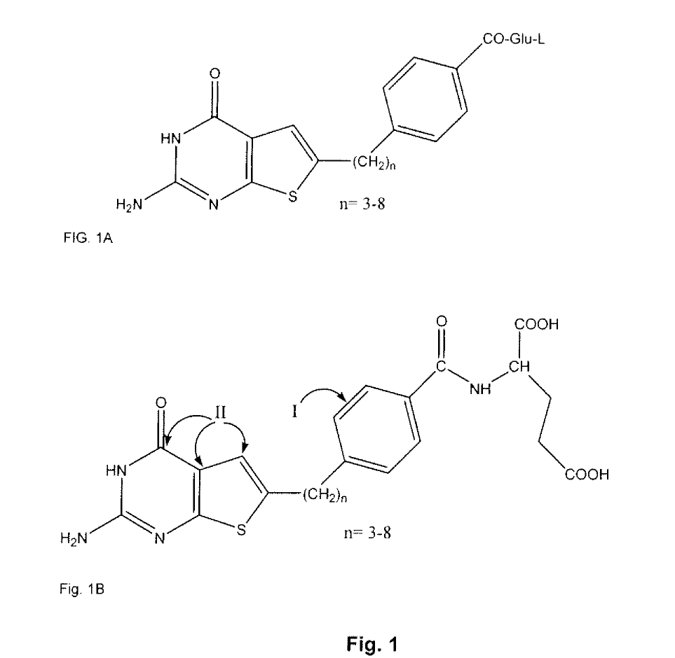

The compounds useful in inhibiting GARFTase and/or AICARFTase in a cancerous tumor of a patient may comprise the fused cyclic pyrimidine shown in FIG. 1(a) and (b), where n=4-8 alkyl chain carbons between the major ring groups, I and II. The compound is preferably effective to selectively target a FR cancerous tumor, where due to the use of long chain carbons, n=4-8, the fused cyclic pyrimidine targets primarily cancerous tumors which contain FR to inhibit GARFTase and AICARFTase within the tumors.

The distance, orientation and location of the side chain p-aminobenzoyl-L-glutamate moiety with respect to the pyrimide ring are important for biological activity; hence, n=4-8 in FIGS. 1(a) and (b) provide surprisingly unique results. While not wishing to be bound by theory, it is hypothesized that the fused cyclic pyrimidine acts as both carrier and cancer treating agent. Conjugation of a separate cancer treating agent to the fused cyclic pyrimidine and cleavage to release a cytotoxic drug is not required.

One embodiment of the present invention relates to compounds that are selective chemotherapeutic agents which selectively target folate receptors (FR) of cancerous tumor cells and inhibit GARFTase and/or AICARFTase contained in the cells, particularly types of ovarian cancer cells. Such compounds comprise fused cyclic pyrimidines having a long chain CH₂ group between cyclic groups.

Such compounds selectively target folate receptors (“FR”), particularly FR—alpha of cancerous tumor cells. The compounds also inhibit glycinamide ribonucleotide formyltransferace enzyme (GARFTase) and/or aminoimidazole carboxamid ribonucleotide formyltransferace enzyme (AICARFTase) in tumor cells. The compounds are effective to selectively penetrate inside of the cancerous tumor cells.

The invention will be more fully understood by review of the drawings in view of the following detailed description of the invention, and the claims appended thereto.

The compounds of the foregoing formula generally display at least one of the following properties properties: 1) inhibition of FR-alpha and beta cancerous tumors, 2) a lack of appreciable uptake by the RFC; 3) ability to act itself as a cancer treating agent; 4) ability to penetrate cancerous tumors having folate receptors; 5) ability to function as a substrate of folylpolyglutamate synthetase (FPGS) thereby being trapped in tumor cells; and 6) inhibition of GARFTase and/or AICARFTase.

The compounds selectively target cancers with certain receptors, and are non-toxic. These fused cyclic pyrimidines are taken into the tumor cells.

Selectivity of the fused cyclic pyrimidine is made possible since most normal cells do not have FRs. FR-alpha is the most widely expressed receptor isoform in adult tissue. FR-alpha occurs at the apical (i.e., luminal) surface of epithelial cells where it is not supplied by folate in the circulation and does not take it up into the cell.

The fused cyclic pyrimidine where n=4-8 has a particular affinity for the receptors such as FR or FR-alpha or FR-beta which are mainly present on the surface of cancerous tumor cells and not other types of folate transport systems that are more predominant on the surface of normal cells. In other words, the fused cyclic pyrimidine of this invention having long chain CH₂ where n=4-8, preferably is not taken up to an appreciable degree by the reduce folate carrier (RFC) system. FR-alpha and beta receptors are generally not expressed in normal cells. The fused cyclic pyrimidine stays inside of the cancerous tumor cell for an adequate amount of time to kill the tumor cell. This occurs by way of polyglutamylation and the multi ionic form of the fused cyclic pyrimidine itself inside of the tumor cell. The fused cyclic pyrimidine also disrupts the replication process of the cancerous tumor cell, thereby inhibiting the growth of FR-alpha expressing cancerous tumor cells.

The foregoing embodiments are enabled by way of a glycinamide ribonucleotide formyltransferase (“GARFTase”) inhibition and/or AICARFTase inhibition. GARFTase is an enzyme which is essential to DNA synthesis of normal and cancerous tumor cells.

Here the fused cyclic pyrimidine itself has a high affinity for the FR-alpha receptors which are overexpressed on the surface of cancerous tumor cells. The fused cyclic pyrimidine passing into the cancerous tumor cells inhibits GARFTase and/or AICRAFTase activity and inhibits DNA synthesis. Accordingly, the targeted tumor cells which overexpress FR-alpha are prevented from replicating and are killed.

In a preferred embodiment, the fused cyclic pyrimidine has a significantly greater affinity for FR-alpha expressing cells compared with cells that do not express FR-alpha. Accordingly, the fused cyclic pyrimidine would have a greater affinity for cells which overexpress FR-alpha (i.e., certain cancerous tumor cells as described in more detail above) but also has an affinity for FR-beta cells.

A further embodiment of the present invention provides a compound of the formula:

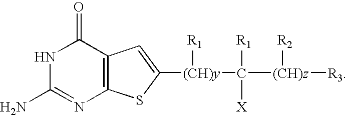

In another embodiment of the present invention, a compound of the formula:

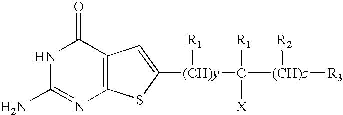

wherein

R1 is H, trifluoromethyl, trifluoromethyl ketone, formyl, methyl alcohol, methylamine or a bond;

X is an aroyl-L-glutamate group or H, wherein if X is H, R2 is an aroyl-L-glutamate group and if X is an aroyl-L-glutamate group, R2 is H or a bond;

R3 is H trifluoromethyl, trifluoromethyl ketone, formyl, methyl alcohol or methylamine;

y is an integer between 0 and 6; and

z is an integer between 1 and 7, wherein the sum total of y and z is equal to or less than 7, and when the sum total of y and z is 1 or 2, X must be an aroyl-L-glutamate group is provided.

The term aroyl, such as for example when used within the term aroyl-L-glutamate, refers to heteroaroyl, benzoyl, napthoyl, thiophenoyl, furophenoyl, pyrroyl, and any other aroyl as that term would be understood by one skilled in the art, including substituted or unsubstituted aroyls. The substitutions on the aroyl group, including the location of L-glutamate on the aroyl, may be in the para, meta or ortho orientations.

In one embodiment, the aroyl-L-glutamate group is p-benzoyl-L-glutamate. In another embodiment, the aroyl-L-glutamate group is 2,5-thienoyl-L-glutamate. In a further embodiment, the aroyl-L-glutamate group is 2,5-pyrroyl-L-glutamate.

For example, in some aspects, the invention is directed to a pharmaceutical composition comprising a compound, as defined above, and a pharmaceutically acceptable carrier or diluent, or an effective amount of a pharmaceutical composition comprising a compound as defined above.

The compounds of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder: comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjutants, protease inhibitors, or other compatible drugs or compounds where such combination is seen to be desirable or advantageous in achieving the desired effects of the methods described herein.

This invention and embodiments illustrating the method and materials used may be further understood by reference to the following non-limiting examples.

1. A compound of the formula: 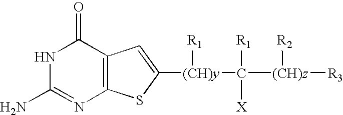
wherein
R1 is H, trifluoromethyl, trifluoromethyl ketone, formyl, methyl alcohol, methylamine or a bond;
X is an aroyl-L-glutamate group or H, wherein if X is H, R2 is an aroyl-L-glutamate group and if X is an aroyl-L-glutamate group, R2 is H or a bond;
R3 is H, trifluoromethyl, trifluoromethyl ketone, formyl, methyl alcohol or methylamine;
y is an integer between 0 and 6; and
z is an integer between 1 and 7, wherein the sum total of y and z is equal to or less than 7, and when the sum total of y and z is 1 or 2, X must be an aroyl-L-glutamate group.

2. A pharmaceutical composition comprising a therapeutically effective amount of a compound of the formula: 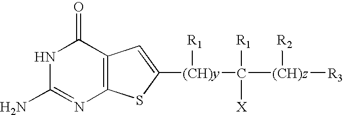
wherein
R1 is H, trifluoromethyl, trifluoromethyl ketone, formyl, methyl alcohol, methylamine or a bond;
X is an aroyl-L-glutamate group or H, wherein if X is H, R2 is an aroyl-L-glutamate group and if X is an aroyl-L-glutamate group, R2 is H or a bond;
R3 is H, trifluoromethyl, trifluoromethyl ketone, formyl, methyl alcohol or methylamine;
y is an integer between 0 and 6; and
z is an integer between 1 and 7, wherein the sum total of y and z is equal to or less than 7, and when the sum total of y and z is 1 or 2, X must be an aroyl-L-glutamate group.

3. A compound effective in inhibiting GARFTase and/or AICARFTase in a cancerous tumors of a patient comprising the formula: 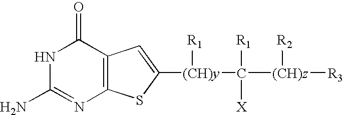
wherein
R1 is H, trifluoromethyl, trifluoromethyl ketone, formyl, methyl alcohol, methylamine or a bond;
X is an aroyl-L-glutamate group or H, wherein if X is H, R2 is an aroyl-L-glutamate group and if X is an aroyl-L-glutamate group, R2 is H or a bond;
R3 is H, trifluoromethyl, trifluoromethyl ketone, formyl, methyl alcohol or methylamine;
y is an integer between 0 and 6; and
z is an integer between 1 and 7, wherein the sum total of y and z is equal to or less than 7, and when the sum total of y and z is 1 or 2, X must be an aroyl-L-glutamate group.

4. The compound of claim 1 wherein said compound is selective for receptors selected from the group consisting of FR-alpha, FR-beta and mixtures thereof associated with cancerous tumors.

5. The compound of claim 1 wherein said compound is not significantly taken up by a tissue or a cell using the RFC system.

6. The compound of claim 1 wherein said compound requires no separate cancer treating agent or conjugation to a separate cytotoxic agent.

7. The compound of claim 1 wherein said compound targets ovarian cancer tumors.

8. The compound of claim 1 wherein said compound targets at least one advanced stage cancerous tumor.

9. The compound of claim 1 wherein said compound targets at least one platinum resistant cancerous tumor.

10. The compound of claim 1 wherein said compound targets at least one carboplatin resistant cancerous tumor.

11. The compound of claim 1 wherein said compound targets at least one paclitaxel resistant cancerous tumor.

12. The compound of claim 1 wherein said compound targets at least one docitaxel resistant cancerous tumor.

13. The compound of claim 1 wherein compound is polyglutamylated by folypoly-gamma glutamate synthetase.

14. The compound of claim 1 wherein said compound targets cancerous tumors selected from the group consisting of ovarian, endometrial, kidney, lung, mesothelioma, breast, and brain tumors.

15. The compound of claim 1 wherein said compound is tolerable in vivo.

16. The compound of claim 1 wherein the sum total of y and z is 1.

17. The compound of claim 1 wherein the sum total of y and z is 2.

18. The compound of claim 1 wherein the sum total of y and z is 3.

19. The compound of claim 1 wherein the sum total of y and z is 4.

20. The compound of claim 1 wherein the sum total of y and z is 5.

21. The compound of claim 1 wherein the sum total of y and z is 6.

22. The compound of claim 1 wherein the sum total of y and z is 7.

23. The compound of claim 1, wherein the aroyl-L-glutamate group is selected from p-benzoyl glutamate, 2,5-thienoyl glutamate and 2,5-pyrroyl glutamate.

24. The compound of claim 2, wherein the aroyl-L-glutamate group is selected from p-benzoyl glutamate, 2,5-thienoyl glutamate and 2,5-pyrroyl glutamate.

25. The compound of claim 3, wherein the aroyl-L-glutamate group is selected from p-benzoyl glutamate, 2,5-thienoyl glutamate and 2,5-pyrroyl glutamate.
